(19) United States
(12) Patent Application Publication
(10) Pub. No.: US 2024/0402190 A1
(43) Pub. Date: Dec. 5, 2024

(54) DIAGNOSTIC BIOMARKERS FOR LIMB-GIRDLE MUSCULAR DYSTROPHY TYPE 2H AND USE THEREOF

(71) Applicant: THE CATHOLIC UNIVERSITY OF KOREA INDUSTRY ACADEMIC COOPERATION FOUNDATION, Seoul (KR)

(72) Inventors: Eun Hui LEE, Seoul (KR); Jun Hee CHOI, Seoul (KR)

(21) Appl. No.: 18/594,798

(22) Filed: Mar. 4, 2024

(30) Foreign Application Priority Data
Mar. 7, 2023 (KR) 10-2023-0029913

(51) Int. Cl. G01N 33/68

(52) U.S. Cl. CPC G01N 33/6887; G01N 33/6851; G01N 2800/2878

(57) ABSTRACT

The present invention relates to a composition for diagnosing limb-girdle muscular dystrophy type 2H, a method of providing information for diagnosis, and a method of screening a drug for the treatment of limb-girdle muscular dystrophy type 2H.

### CROSS-REFERENCE TO RELATED APPLICATION

This application claims priority to and the benefit of Korean Patent Application No. 2023-0029913, filed on Mar. 7, 2023, the disclosure of which is incorporated herein by reference in its entirety.

### SEQUENCE LISTING

The text of the computer readable sequence listing filed Mar. 4, 2024, titled “G24U11C0092P_US.xml” created Mar. 4, 2024, having a file size of 4,500 bytes, is hereby incorporated by reference in its entirety.

### BACKGROUND

### 1. Field of the Invention

The present invention relates to a diagnostic marker for limb-girdle muscular dystrophy type 2H or a use thereof.

### 2. Discussion of Related Art

Muscular dystrophy (MD) is a genetic disease that causes progressive weakness and degeneration of skeletal muscles that control body movement and posture. Some forms of muscular dystrophy occur in infancy or childhood, while other forms may occur in middle age or later, or very rarely, may not appear at all. When muscular dystrophy does not appear, it can be passed on to descendants, and the descendants will be diagnosed with the disease without any preparation. The disorders differ in terms of the distribution and severity of muscle weakness (some forms of muscular dystrophy also affect the cardiac muscle), the age of onset, the rate of progression, or inheritance patterns. Types of muscular dystrophy mainly include Duchenne muscular dystrophy, Becker muscular dystrophy, limb-girdle muscular dystrophy, facioscapulohumeral muscular dystrophy, oculopharyngeal muscular dystrophy, and the like.

Among them, limb-girdle muscular dystrophy (LGMD) ranks 3^{rd} to 5^{th} in frequency of occurrence among muscular dystrophies (slightly different depending on the scope of investigation, such as year or region). LGMD appears to varying degrees from person to person with regard to the age of onset, the area of muscle weakness, the cardiac and respiratory involvement in some patients, the rate of progression, and the severity. Limb-girdle muscular dystrophy may begin in childhood, adolescence, young adulthood, or even later. Both genders are equally affected. Limb-girdle muscular dystrophy causes weakness in the shoulders and pelvic girdle, and sometimes causes weakness in the surrounding muscles of the upper legs and arms over time. Weakness in the legs usually appears before weakness in the arms. Facial muscles are generally unaffected or minimally affected. As the condition progresses, patients have problems in walking and, over time, will need to use a wheelchair. In most cases, people can do their daily activities only with the help of others. When the shoulder and arm muscles are involved, it causes difficulties in raising the arms above the head and lifting objects. Some types of limb-girdle muscular dystrophy may be accompanied by pathological cardiac and respiratory abnormalities. There are several forms of limb-girdle muscular dystrophy, which are classified according to the genetic defects involved therein. Limb-girdle muscular dystrophy has an autosomal inheritance pattern, and there is currently no known cure for limb-girdle muscular dystrophy.

Mutations in the tripartite motif-containing protein 32 (TRIM32) gene cause limb-girdle muscular dystrophy type 2H (LGMD2H), which is a rare muscular dystrophy. In particular, it has been known that most LGMD2H cases are caused by mutations occurring in the NHL repeat region present in the TRIM32 protein. For example, four mutations of TRIM32 are known in the C-terminal NHL domain: D487N (the third NHL repeat), R394H (the second NHL repeat), T520TfsX13 (the fifth NHL repeat), and D588del (the fifth NHL repeat). However, the detailed mechanism of pathogenesis or treatment method has not been known at all.

To date, there are no effective drugs for muscular dystrophy, and only some symptomatic therapies such as steroid administrations exist. Steroid administrations have an effect of prolonging the walking period temporarily, but they cannot stop or reverse the progression of the disease, and there is a major problem of having to endure the side effects from long-term use. In addition, gene therapy has been developed, but it has problems in that only approximately 10 to 13% of patients are eligible for exon skipping gene therapy, weekly subcutaneous or intravenous administration is required, and it is impossible to expect treatment when it is accompanied by cardiomyopathy due to poor infiltration into myocardial cells. Also, there is a problem that nonsense mutation treatment can be applied to only a very small number of patients (approximately 11 to 14%). In case of Duchenne muscular dystrophy, gene therapy using viruses has the problem in that the efficiency of gene transfer is very low because the size of a dystrophin gene (cDNA having a length of approximately 14 kb) is very large compared to the size of the vector. To overcome this problem, a microdystrophin in which only 4 or 5 of the 24 spectrin-like repeats remain in the middle of dystrophin was developed, but there are problems such as side effects of immune responses due to the AAV vector or very limited expression of dystrophin. Therefore, there is an urgent need to develop a more fundamental treatment without any side effects in order to treat muscular dystrophy. Because these methods have never been attempted for limb-girdle muscular dystrophy type 2H, there has been no therapeutic options for limb-girdle muscular dystrophy type 2H.

Accordingly, the present inventors have identified the mechanism of action for diagnosis and treatment of limb-girdle muscular dystrophy type 2H. Therefore, the present invention has been completed based on these findings.

### SUMMARY OF THE INVENTION

The present invention is directed to providing a composition for diagnosing limb-girdle muscular dystrophy type 2H.

The present invention is also directed to providing a method of providing information for the diagnosis of limb-girdle muscular dystrophy type 2H.

The present invention is also directed to providing a method of screening a drug for the treatment of limb-girdle muscular dystrophy type 2H.

The present inventors confirmed the expression levels of Orai1, TRPC3, and TRPC6, which are key proteins that mediate external calcium influx as a calcium transport pathway required for the contraction of skeletal muscles in myotubes, and found that the expression of Orai1 and TRPC3 decreases while the expression of TRPC6 increases when NHL-Del is expressed in the myotubes.

According to an aspect of the present invention, there is provided a composition for diagnosing limb-girdle muscular dystrophy type 2H, which includes an agent for measuring an expression level of the mRNA or protein of one or more genes selected from the group consisting of Orai1, TRPC3, and TRPC6.

According to another aspect of the present invention, there is provided a method of providing information for the diagnosis of limb-girdle muscular dystrophy type 2H, which includes: measuring an expression level of the mRNA or protein of one or more genes selected from the group consisting of Orai1, TRPC3, and TRPC6 in a biological sample isolated from a subject.

According to still another aspect of the present invention, there is provided a method of screening a drug for the treatment of limb-girdle muscular dystrophy type 2H, which includes: treating a biological sample, which is isolated from a subject suspected of having limb-girdle muscular dystrophy type 2H, with a candidate material, and judging whether the candidate material increases or decreases the expression of the mRNA or protein of one or more genes selected from the group consisting of Orai1, TRPC3, and TRPC6 in skeletal muscle cells.

### BRIEF DESCRIPTION OF THE DRAWINGS

FIG. 1A shows a schematic diagram of wild-type TRIM32 or NHL repeats. In FIG. 1A, numbers represent amino acids. FIG. 1B shows the expression of a GST-NHL protein (left), the degree of purification of GST-NHL (middle), and a binding reaction of a triad sample with GST-NHL (right), as determined by Coomassie Blue staining. FIGS. 1C to 1G show the MALDI-TOF/TOF spectrum results for bands 1 to 5. FIG. 1H shows the results of co-immunoprecipitation using a triad sample obtained from rabbit skeletal muscle and a SERCA1a antibody, and immunoblotting analysis of the obtained sample using SERCA1a and TRIM32 antibodies (top); and shows the results of co-immunoprecipitation using a lysate sample of mouse myotubes and a SERCA1a antibody, and immunoblotting analysis of the obtained sample using SERCA1a and TRIM32 antibodies (bottom).

FIG. 2A shows a schematic diagram of wild-type TRIM32 or NHL-Del. In FIG. 2A, numbers represent amino acids. FIG. 2B shows the results of determining the expression of a vector (a vector control), wild-type TRIM32, or NHL-Del in myotubes using immunochemistry (white line: 100 μm). FIG. 2C shows the results of measuring the width of the thickest part of myotubes. FIG. 2D shows the results of determining the expression levels of MyoD and myogenin proteins, which are differentiation marker proteins, and a-actin, which is a structural protein in all cells and is used as loading control, through immunoblotting analysis.

FIG. 3 shows the results of performing immunoblotting analysis to compare the expression levels of RyR1, DHPR, SERCA1a, TRIM32 (existing in the art), MG53, and CASQ1, which are key proteins that mediate the contraction and relaxation of skeletal muscle.

FIG. 4A is a bar graph showing the amount of calcium stored in the sarcoplasmic reticulum in mouse myotubes expressing wild-type TRIM32 or NHL-Del. FIG. 4B shows the results of relatively measuring the degree of calcium release from the sarcoplasmic reticulum to the cytosol by treating mouse myotubes with caffeine. FIG. 4C shows the results of measuring the relative amount of calcium released from the sarcoplasmic reticulum to the cytosol by treating the mouse myotubes with KCl.

FIG. 5 shows the results of performing immunoblotting analysis to compare the expression levels of key proteins that mediate the influx of external calcium required for the contraction of skeletal muscle.

### DETAILED DESCRIPTION OF EXEMPLARY EMBODIMENTS

Hereinafter, configurations of the present invention will be described in detail.

In the present invention, the term “tripartite motif-containing protein 32 (TRIM32)” refers to a member of the E3 ubiquitin ligases that are involved in the breakdown of thin filaments (also referred to as actin filaments) such as actin, tropomyosin, troponin, α-actinin, and desmin. TRIM32 is a protein that is composed of an N-terminal RING domain, a B-box domain, a coiled-coil region, and a C-terminal NHL repeat, and is derived from mammals, preferably humans, mice, house rats, rabbits, orangutans, monkeys, hamsters, cats, dolphins, gorillas, and the like. For example, the amino acid sequence of the TRIM32 protein and the sequence of the gene thereof are known as GenBank Accession Nos. NP_001093149.1 and NM_001099679.2, respectively.

In the present invention, the term “NHL repeat” refers to an amino acid sequence found in a large number of eukaryotic and prokaryotic proteins, named after ncl-1, HT2A, and lin-41, and has been used for a long time. In particular, the NHL repeat of the present invention refers to an NHL repeat present in TRIM 32.

In the present invention, the term “NHL-Del” refers to a mutation in TRIM32, that is, a mutation lacking the NHL repeat of TRIM32.

In the present invention, the term “sarcoplasmic/endoplasmic reticulum Ca²⁺-ATPase 1a (SERCA1a)” is a type of Ca²⁺-pump protein which consumes ATP and uptakes calcium present in the cytosol into the sarcoplasmic reticulum (SR). The above process is a key process that initiates a process of returning to the normal state (i.e., a relaxed state) after the muscle contracts.

In order for skeletal muscle to initiate and sustain its contraction, a large amount of calcium (several micromolar level) is required in the cytosol of skeletal muscle cells (i.e., skeletal myotubes). This calcium is transported by releasing calcium stored in the sarcoplasmic reticulum into the cytosol through ryanodine receptor 1 (RyR1), which is an internal calcium channel. After contraction, a process of returning a large amount of calcium present in the cytosol for contraction into the sarcoplasmic reticulum is essential for the relaxation of skeletal muscle. In this case, SERCA1a (la indicates the main type of skeletal muscle SERCA) serves as a Ca²⁺-pump protein for calcium to return to the sarcoplasmic reticulum. That is, SERCA1a consumes ATP and use its power to uptake cytosolic calcium back into the sarcoplasmic reticulum. Therefore, because the role of SERCA1a is very important for the relaxation of skeletal muscle as well as the next contraction of skeletal muscle, SERCA1a is an essential key protein required during skeletal muscle contraction and relaxation.

The present inventors found that TRIM32 binds to SERCA1a to increase the amount of calcium in the sarcoplasmic reticulum for skeletal muscle contraction and increase intracellular calcium transport for skeletal muscle contraction. The interaction between TRIM32 and SERCA1a occurs through the NHL repeat of TRIM32. Therefore, mutations in TRIM32, specifically mutations in the NHL repeat of TRIM32 (e.g., NHL-Del), are unable to interact with SERCA1a.

Therefore, whether the interaction between TRIM32 or NHL repeat and SERCA1a increases or decreases may be used to screen or diagnose drugs for the treatment of limb-girdle muscular dystrophy type 2H.

In the present invention, the term “calcium release-activated calcium channel protein 1 (Orai1)” is a key protein for store-operated calcium (Ca²⁺) entry (SOCE), as a cation-selective ion channel that is more selective to Ca²⁺ ion than any other cations. Orai1 is activated when the internal calcium stores (i.e., the sarcoplasmic reticulum) are depleted. The reduced calcium concentration in the sarcoplasmic reticulum is sensed by the STIM1 protein. In this case, STIM1 activates Orai1, which is present on the cell membrane, through protein-protein interactions, and calcium is transferred from the outside of the cell to the inside of the cells (i.e., cytosol) through Orai1. Information on the gene and protein of Orai1 is registered in NCBI (NM_032790.3, NP_116179.2).

In the present invention, the terms “transient receptor potential channel 3 (TRPC3)” and “transient receptor potential channel 6 (TRPC6)” are cation channels that regulate calcium influx in the cells by detecting the depletion of Ca²⁺ stores to cause the calcium influx from the outside of the cells into the cytosol. The information on TRPC3 and TRPC6 genes and proteins is registered in NCBI (TRPC3: NM_003305, NP_003296; TRPC6: NM_004621, NP_004612).

The present inventors confirmed that the expression of Orai1 and TRPC3 decreases while the expression of TRPC6 increases when NHL-Del was expressed in myotubes.

Therefore, the Orai1, TRPC3, or TRPC6 gene may be used to diagnose limb-girdle muscular dystrophy type 2H by measuring the expression level of the mRNA or protein of the Orai1, TRPC3, or TRPC6 gene.

The present invention provides a composition for diagnosing limb-girdle muscular dystrophy type 2H, which includes an agent for measuring the expression level of the mRNA or protein of one or more genes selected from the group consisting of Orai1, TRPC3, and TRPC6.

In the present invention, the term “diagnosis” refers to a series of actions that determine the presence or characteristics of a disease in a subject. In the present invention, the diagnosis may include an action of determining whether a disease has occurred. Also, in the present invention, the diagnosis may include an action of checking the risk of developing a disease.

In the present invention, the term “subject” refers to an individual whose risk of developing a disease is to be determined, and more specifically, any mammal, for example, humans and primates, as well as livestock such as cattle, pigs, sheep, horses, dogs, and cats, but the present invention is not limited thereto. Preferably, the subject may be a human.

According to one embodiment of the present invention, the measurement of protein interaction or expression level may be performed by measuring the interaction or level in skeletal muscle cells.

According to one embodiment of the present invention, the agent for measuring the mRNA level of the gene may be a probe or primer that specifically binds to the gene. The measurement may be performed by one or more methods selected from the group consisting of polymerase chain reaction, real-time RT-PCR, reverse transcription polymerase chain reaction, competitive polymerase chain reaction (competitive RT-PCR), a nuclease protection assay (RNase, S1 nuclease assay), an in situ hybridization method, a nucleic acid microarray, Northern blot, DNA chips, multiplex PCR, or ddPCR, but the present invention is not limited thereto.

In the present invention, the term “primer” refers to a single-stranded oligonucleotide that may act as an initiation site for template-directed DNA synthesis under suitable conditions (i.e., four different nucleoside triphosphates and a polymerization enzyme) at a suitable temperature and in a suitable buffer. The appropriate length of the primer may vary depending on various factors such as temperature and the intended use of the primer. Also, the sequence of the primer does not need to be completely complementary to a partial sequence of the template, and it is sufficient to have sufficient complementarity within the range where the primer can hybridize with the template to perform its original function. Therefore, the primer in the present invention does not need to have a perfectly complementary sequence to the nucleotide sequence of the template gene, and it is sufficient to have sufficient complementarity within the range where the primer can hybridize to the gene sequence to perform its function.

In the present invention, the term “probe” refers to a nucleic acid fragment capable of binding to a specific nucleic acid in a sequence-specific manner. The probe may be a nucleic acid fragment, such as RNA or DNA, that is as short as a few bases or as long as several hundred bases, and is labeled so that the presence or absence of a specific nucleic acid can be checked. Probes may be constructed in the form of oligonucleotide probes, single-stranded DNA probes, double-stranded DNA probes, RNA probes, and the like.

Primers or probes may be chemically synthesized using a phosphoramidite solid support method, or other well-known methods. These nucleic acid sequences may also be modified using many means known in the art. Non-limiting examples of such modifications include methylation, capping, substitution of a native nucleotide with one or more homologs, and modifications between nucleotides, such as modifications with uncharged linkages (e.g., methyl phosphonates, phosphotriesters, phosphoramidates, carbamates, and the like) or charged linkages (e.g., phosphorothioate, phosphorodithioate, and the like).

In the present invention, the primer may be used to measure the mRNA level by hybridizing to the Orai1, TRPC3, and/or TRPC6 gene sequence to amplify a DNA fragment containing a gene region. Also, in the present invention, hybridization may be performed using probes complementary to the Orai1, TRPC3, and/or TRPC6 gene sequences of the present invention, and the mRNA level of the gene may be measured based on the hybridization.

According to another embodiment of the present invention, the agent for measuring the protein level may be an antibody, an aptamer, an avimer (short for avidity multimer), or a peptidomimetic specific for the protein. The measurement may be performed using one or more methods selected from the group consisting of Western blot, an enzyme-linked immunosorbent assay (ELISA), a radioimmunoassay (RIA), radioimmunodiffusion, immunoelectrophoresis, tissue immunostaining, an immunoprecipitation assay, a complement fixation assay, FACS, mass spectrometry, or protein chips, but the present invention is not limited thereto.

In the present invention, the term “antibody” is a term known in the art and refers to a specific protein molecule directed to an antigenic site. The form of the antibody of the present invention is not particularly limited and includes polyclonal antibodies, monoclonal antibodies, or functional fragments having antigen binding properties as well as all immunoglobulin antibodies. Furthermore, the antibodies of the present invention also include special antibodies such as humanized antibodies and the like. The functional fragment of the antibody molecule refers to a fragment that possesses at least an antigen-binding function and includes Fab, F(ab′), F(ab′)2, ScFv, and the like.

In the present invention, the term “aptamer” refers to a nucleic acid that may specifically and strongly bind to a specific molecule while maintaining a stable tertiary structure. Because of its specific binding function, the aptamer is compared to antibodies and has been evaluated as an alternative technology to antibodies.

In the present invention, the interaction between the TRIM32 and/or NHL repeat and SERCA1a may be determined by applying techniques commonly used in the art to confirm protein-protein or protein-compound reactions. For example, a fluorescence assay, a yeast two-hybrid method, screening for phage display peptide clones that bind to the TRIM32 and/or NHL repeat or SERCA1a, immunoprecipitation, co-immunoprecipitation, reverse co-immunoprecipitation, GST-fusion protein pulldown, affinity chromatography, crosslinking, immunohistochemistry, screening methods such as high throughput screening using natural and chemical libraries, drug hit HTS, or cell-based screening, and the like may be used, but the present invention is not limited thereto.

The composition for diagnosing limb-girdle muscular dystrophy type 2H of the present invention may be provided in the form of a diagnostic kit. The kit may be manufactured by conventional methods known to those skilled in the art.

Also, the kit of the present invention may be an agent for measuring the expression level of the mRNA or protein of one or more genes selected from the group consisting of Orai1, TRPC3, and TRPC6, and may also include one or more other component compositions, solutions or devices suitable for the analysis method.

According to one embodiment of the present invention, the kit may be an RT-PCR kit, a microarray chip, or a microfluidic chip kit.

According to one embodiment, the kit of the present invention may be a kit containing the essential elements required to perform PCR, and may further include each primer pair capable of amplifying a nucleic acid, a test tube or another suitable container, a reaction buffer (various pH values and magnesium concentrations), deoxynucleotides (dNTPs), enzymes such as Taq-polymerase and reverse transcriptase, DNase, an RNAse inhibitor, DEPC-water, sterilized water, and the like.

According to another embodiment, the kit of the present invention may be a kit for diagnosing a respiratory disease, which includes the essential elements required to perform DNA chip analysis. Preferably, the kit of the present invention may be a microarray chip or microfluidic chip kit. The DNA chip kit includes a substrate to which a primer or probe specific for the mRNA of a gene is attached, and the substrate may include a nucleic acid corresponding to a quantitative control gene or a fragment thereof. In such a DNA chip kit, nucleic acids are arranged uniformly on the chip surface, which makes it possible to perform a massively parallel assay by causing multiple hybridization reactions between the nucleic acids on the DNA chip and the complementary nucleic acids contained in the solution treated on the chip surface.

Hybridization of nucleic acids and detection of hybridization results on microarrays are well known in the art. The hybridization results may be detected, for example, by labeling a nucleic acid sample with a labeling material capable of generating a detectable signal, which includes a fluorescent substance such as Cy3 and Cy5, hybridizing the labeling material on a microarray, and then detecting the signal generated from the labeling material.

A microfluidic chip is a microfluidic device that uses microfluidic control technology to determine and analyze the interaction of an analyte included in a fluid sample with a biological material, cell, tissue, or detection device on the chip, and may include a miniaturized and compartmentalized space in which processes such as probe/target hybridization, nucleic acid amplification, and capillary electrophoresis reactions may occur.

When the kit of the present invention is a kit for measuring the expression level of a protein, in order to immunologically detect an antibody, the kit may include a substrate, an appropriate buffer solution, a secondary antibody labeled with a chromogenic enzyme or a fluorescent substance, a chromogenic substrate, and the like. In this case, a nitrocellulose membrane, a 96-well plate synthesized from a polyvinyl resin, a 96-well plate synthesized from a polystyrene resin, and a glass slide made of glass, and the like may be used as the substrate, and peroxidase or alkaline phosphatase may be used as the chromogenic enzyme. Also, fluorescein isothiocyanate (FITC), rhodamine B isothiocyanate (RITC), and the like may be used as the fluorescent substances, and 2,2′-azino-bis(3-ethylbenzothiazoline-6-sulfonic acid (ABTS), o-phenylenediamine (OPD), or tetramethylbenzidine (TMB) may be used as the chromogenic substrate solution.

For genotyping of the present invention, sequencing, hybridization analysis using a microarray and the like, amplification using PCR and the like may be used.

The genotyping of the present invention may be performed by genetic sequencing. For example, known methods such as sequencing, mini-sequencing, automated sequencing, TaqMan analysis, pyrosequencing, allele-specific PCR, dynamic allele-specific hybridization (DASH), PCR-restriction fragment length polymorphism (PCR-RELP), PCR-single strand conformation polymorphism (PCR-SSCP), PCR-specific sequence oligonucleotide (PCR-SSO), hybridization using a microarray, primer extension, Southern blot hybridization, dot hybridization, allele specific oligonucleotide (ASO) hybridization in which PCR-SSO is combined with dot hybridization, rolling circle amplification (RCA), high-resolution melting (HRM), or matrix-assisted laser desorption ionization-time of flight mass spectrometry (MALDI-TOF/MS), and the like may be used, but the present invention is not limited thereto.

Furthermore, the results of the analysis may be statistically processed using statistical analysis methods commonly used in the art. For example, the analysis results may be analyzed using variables, such as continuous variables, categorical variables, odds ratios, and 95% confidence intervals, obtained through a Student's t-test, a Chi-square test, linear regression line analysis, multiple logistic regression analysis, and the like.

The present invention provides a method of providing information for the diagnosis of limb-girdle muscular dystrophy type 2H, which includes: measuring the expression level of mRNA or protein of one or more genes selected from the group consisting of Orai1, TRPC3, and TRPC6 in a biological sample isolated from a subject.

All contents described above in relation to the diagnostic composition and kit may be applied directly or mutatis mutandis to the method.

In the present invention, the term “biological sample” includes tissue, cells, whole blood, serum, plasma, saliva, sputum, cerebrospinal fluid, urine, or the like obtained from a subject, but the present invention is not limited thereto.

According to one embodiment of the present invention, when compared to the normal control according to the above information provision method, it may be judged that the subject has developed limb-girdle muscular dystrophy type 2H or is at high risk of developing limb-girdle muscular dystrophy type 2H when the expression level of Orai1 and/or TRPC3 decreases compared to the expression level in the normal control sample: or when the expression level of TRPC6 increases compared to the expression level in the normal control sample.

The present invention provides a method of screening a drug for the treatment of limb-girdle muscular dystrophy type 2H.

According to the present invention, the candidate material may be a material presumed to have the potential as a medicine that increases the expression of the mRNA or protein of the Orai1 and/or TRPC3 genes and decreases the expression of the mRNA or protein of the TRPC6 gene in skeletal muscle cells according to a conventional selection method, or may be a randomly selected individual nucleic acid, protein, peptide, other extract, natural product, compound, or the like.

All contents described above in relation to the diagnostic composition, the kit, and the method of providing information for diagnosis may be applied directly or mutatis mutandis to the method.

A candidate material that increases the expression of the mRNA or protein of the Orai1 and/or TRPC3 gene and decreases the expression of the mRNA or protein of the TRPC6 gene in skeletal muscle cells obtained through the screening method of the present invention may be a candidate material for treatment of limb-girdle muscular dystrophy type 2H. In other words, when a candidate material increases the expression of the mRNA or protein of the Orai1 and/or TRPC3 gene and decreases the expression of the mRNA or protein of the TRPC6 gene in the skeletal muscle cells, the candidate material may be judged to be a material capable of treating limb-girdle muscular dystrophy type 2H.

Such a candidate material for the treatment of limb-girdle muscular dystrophy type 2H will serve as a leading compound in the subsequent development of a therapeutic agent for limb-girdle muscular dystrophy type 2H. In this case, a new therapeutic agent for limb-girdle muscular dystrophy type 2H may be developed by modifying and optimizing a structure of the leading compound so that the leading compound can have an effect of increasing the expression of the mRNA or protein of the Orai1 and/or TRPC3 gene and decreasing the expression of the mRNA or protein of the TRPC6 gene in skeletal muscle cells.

Advantages and features of the present invention and methods of accomplishing the same will be apparent with reference to embodiments described below in detail. However, it should be understood that the present invention is not limited to the embodiments disclosed below and may be implemented in various different forms. In this specification, the embodiments of the present invention are merely provided to make the disclosure of the present invention complete and to fully inform the scope of the present invention to those skilled in the art to which the present invention pertains. Accordingly, the present invention is defined only by the scope of the claims.

### Experimental Method

This experiment was conducted in accordance with the regulations and guidelines of the College of Medicine at the Catholic University of Korea (Ethical Approval Regulation 2017-0117-01). The sites where animal work and any surgical intervention were performed were set in accordance with the Guidelines and Policies for Rodent Survival Surgery, the Laboratory Animals Welfare Act, and the Guide for Care and Use of Laboratory Animals approved by the Institutional Animal Care and the Use Committee of the College of Medicine at the Catholic University of Korea. All protocols for animal-related experiments were approved by the Committee of the College of Medicine at The Catholic University of Korea.

### 1-1. Method of Constructing cDNA for GST-Linked NHL Repeat Protein (GST-NHL) and Expressing cDNA as Protein

Using human TRIM32 cDNA (GenBank Accession Code: NM_012210.3) as a sample, PCR was performed using the PCR primers provided in Table 1. The obtained synthesized PCR product was inserted into pGEX-4T-1, and the base sequence was confirmed again through a protein sequencer. The pGEX-4T-1 was transformed into E. coli (DH5α), expressed as a protein, and purified using GST beads.

TABLE 1
PCR primer sequences (Xho I and BamH I) for
constructing cDNA for NHL repeat protein

| | |
|---|---|
| Forward primer | 5′-CGGAATTCATGTTCAATCTTCC-3′ |
| (SEQ ID NO: 1) | |
| Reverse primer | 5′-CCTCGAGTCATGGGGTGGAATATC-3′ |
| (SEQ ID NO: 2) | |

### 1-2. Preparation of Triad Sample and Binding Reaction with GST-NHL

Triad vesicles were obtained from rabbit fast-twitch skeletal muscle (back and leg), and lysed at 4° C. for 4 hours in a lysis solution (1% Triton X-100, 10 mM Tris-HCl, 1 mM Na₃VO₄, 10% glycerol, 150 mM NaCl, 5 mM EDTA, and a protease inhibitor cocktail, pH 7.4) to obtain a triad sample. For binding analysis, GST-NHL was attached to GST beads (Abcam, Cambridge, MA; Cat. No. ab193267), and the triad sample was then mixed therewith. Then, a binding reaction was performed at 4° C. for 8 hours. The proteins attached to the obtained beads were separated on an SDS-PAGE gel, and visualized through Coomassie Brilliant Blue staining.

### 1-3. Protein Identification Through MALDI-TOF/TOF Mass Spectrometry and Database Search

The proteins obtained from the triad sample preparation and the binding reaction with GST-NHL were subjected to protein digestion using trypsin in a gel state. The digested peptide solution was desalted and concentrated using a C18 nanocolumn (100 to 300 nL of 20 to 30 μm-sized POROS reverse-phase R2 material; Thermo Fisher Scientific, Waltham, MA; Cat. No. 1102412) and 1.5 mL of 50% MeOH, 49% H₂O, and 1% formic acid. Thereafter, the peptide mass was analyzed by MALDI-TOF/TOF mass spectrometry (4700 Proteomics Analyzer MALDI-TOF/TOF, Applied Biosystems, Waltham, MA). The analysis results were searched for using the NCBI database, and the identity of the corresponding protein was confirmed.

### 1-4. Co-Immunoprecipitation Method

The triad sample was subjected to co-immunoprecipitation using a SERCA1a antibody, and the resulting product was separated on a 10% SDS-PAGE gel. Thereafter, the proteins separated on the gel were transferred to a polyvinylidene fluoride (PVDF) membrane (at 100 V for 2 hours) and treated with 5% non-fat milk for an hour. Then, the proteins were treated with the corresponding primary antibody (for 3 to 12 hours; a TRIM32 antibody or a SERCA1a antibody), followed by the corresponding secondary antibody (a horseradish peroxidase-conjugated secondary antibody) for 45 minutes. Then, the proteins were visualized and analyzed through a chromogenic reaction (SuperSignal ultrachemiluminescent substrate). To perform an immunoblotting assay using mouse myotubes, the myotubes were lysed at 4° C. for 24 hours in a lysis solution (lysis buffer composition: 1% Triton X-100, 10 mM Tris-HCl (pH 7.4), 1 mM Na₃VO₄, 10% glycerol, 150 mM NaCl, 5 mM EDTA, protease inhibitors (1 μM pepstatin, 1 μM leupeptin, 1 mM phenylmethylsulfonyl fluoride, 20 mg/mL aprotinin, 1 μM trypsin inhibitor)). At this time, in the case of the myotubes differentiated in a 10-cm culture dish, the cells were treated with 300 μL of a lysis solution. The subsequent antibody treatment and chromogenic reaction were performed as described above (a co-immunoprecipitation method using a triad sample).

### 1-5. Construction of cDNA for NHL-Del and Protein Expression

To express NHL-Del in mammalian cells, PCR was performed using human TRIM32 cDNA (GenBank accession code: NM_012210.3) as a sample and using the PCR primers shown in Table 2. The resulting synthesized PCR product was transferred to a pEGFP-N1 vector to construct NHL-Del cDNA for expression in mammalian cells. The base sequence of the constructed cDNA was confirmed again through a gene sequencer, and wild-type TRIM32 for expression in mammalian cells was obtained from Addgene (Cat #69541).

TABLE 2
PCR primer sequences (Xho I and BamH I)
for constructing cDNA for NHL-Del

| | |
|---|---|
| Forward primer | 5′-GACTCAGATCTCGAGGCCACCATG-3′ |
| (SEQ ID NO: 3) | |
| Reverse primer | 5′-CGGGATCCCATTCCTGGAGTG-3′ |
| (SEQ ID NO: 4) | |

### 1-6. Isolation of Skeletal Muscle Satellite Cells and Differentiation Into Myotubes

Skeletal muscle satellite cells were isolated from the skeletal muscles of mice and primarily cultured to obtain myoblasts. During the culture process, the cells were treated with a cell culture medium (F10 Nutrient Mixture composition: 20% FBS, 100 units/mL penicillin, 100 μg/mL streptomycin, 2 mM L-glutamine, and 20 nM basic fibroblast growth factor) and incubated at 37° C. in a 5% CO₂ incubator. A 10-cm or 96-well culture dish was used as the culture dish depending on the purpose, and all the dishes were coated with Matrigel. When the cells proliferated enough to occupy approximately 70% of the culture dish, the culture medium was replaced with a differentiation medium, and differentiation into myotubes was induced (Composition of cell differentiation medium: 5% heat-inactivated horse serum and low-glucose DMEM were used instead of 20% FBS and F-10 Nutrient mixture in the cell culture medium, no bFGF was added, and an 18% CO₂ incubator was used). The completely differentiated cells are referred to as “myotubes,” which were used in the relevant experiments below.

### 1-7. Expression of TRIM32 and NHL-Del in Myotubes

cDNA for TRIM32 and NHL-Del was genetically transfected into immature myotubes on day 3 from the day of differentiation induction for 4 hours (cDNA transfection: each cDNA was in the form of a pEGFP-NI vector, and taking cells cultured in a 10-cm culture dish as an example, 30 μL of FuGENE6 and 20 μg of cDNA were processed together). Further differentiation of the cells transfected with cDNA was induced for 36 hours. The cells in this state are myotubes whose differentiation was completed and simultaneously express TRIM32 and NHL-Del, which were used in the experiments below.

### 1-8. Immunocytochemistry

To confirm the expression of wild-type TRIM32 and NHL-Del in myotubes, immunocytochemistry was performed. Myotubes were fixed with cold methanol for 30 minutes, and this experiment was performed using a GFP primary antibody (1:500) and a cy3-conjugated secondary antibody (1:500, Sigma).

### 1-9. Method of Measuring Width of Myotubes

The length of the thickest part of the myotubes whose differentiation was completed was measured using the ImageJ program.

### 1-10. Immunoblotting Analysis

A sample was separated on an 8%, 10%, or 12% SDS-PAGE gel, and proteins separated on the gel were transferred to a polyvinylidene fluoride (PVDF) membrane (at 100 V for 2 hours) and treated with 5% non-fat milk for an hour. Thereafter, the proteins were treated with the corresponding primary antibody (1:1000), followed by the corresponding secondary antibody (a horseradish peroxidase-conjugated secondary antibody) for 45 minutes. Then, the proteins were visualized and analyzed through a chromogenic reaction (SuperSignal ultrachemiluminescent substrate, Pierce).

### 1-11. Single-Cell Calcium Imaging Technique for Determining Calcium Responses in Myotubes to Thapsigargin (TG), Caffeine, or KCl

Myotubes were treated (incubated) with fluo-4 (5 μM), which is a calcium fluorescent dye (Ca²⁺dye) that emits fluorescence at a different wavelength than before calcium binding when combined with calcium, at a constant temperature of 37° C. for 45 minutes. In this case, the myotubes were treated with an imaging solution (125 mM NaCl, 5 mM KCl, 2 mM KH₂PO₄, 2 mM CaCl₂, 25 mM HEPES, 6 mM glucose, 1.2 mM MgSO₄, 0.05% BSA (Fraction V), pH 7.4). A fluorescence microscope (Nikon x40 oil-immersion objective, NA 1.30, ECLIPSE Ti, Nikon) was used to measure intracellular calcium transport. Changes in fluorescence of the calcium fluorescent dye were transmitted to a computer using a 75-watt Xenon lamp (FSM150Xe, Bentham Instruments, Ltd) and a 12-bit CCD camera (DVC-340M-OO-CL, Digital Video Camera Company) connected to the fluorescence microscope, and analyzed using InCyt Im1 image acquisition and analysis software (v5.29, Intracellular Imaging Inc.). The calcium transport from the sarcoplasmic reticulum (SR) to the cytosol and the concentration of calcium in the cytosol were determined by treatment with thapsigargin, caffeine, and KCl. Then, the values for the peak heights or areas on the graph were statistically processed (showing the same tendency as the statistical processing of values for the areas on the graph).

### 1-12. Data Analysis (Statistical Analysis)

All data was expressed as ±SE by combining data obtained through multiple experiments. The value obtained in the control was set to 1 and expressed as a normalized ratio expressing a relative change in the value. Significant differences were determined using an unpaired t-test or one-way ANOVA-Tukey's post hoc test (GraphPad InStat, v2.04), and the significant differences were indicated when P<0.05 (* or #). The Origin 2019b program was used to plot the graph.

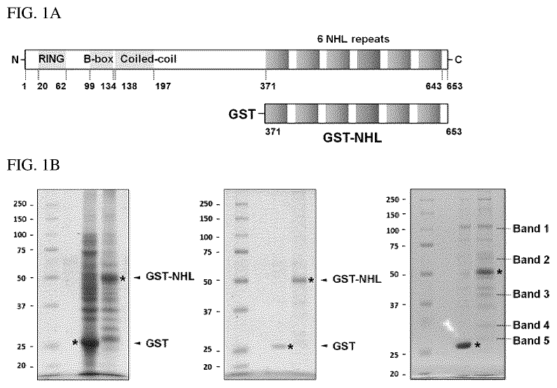

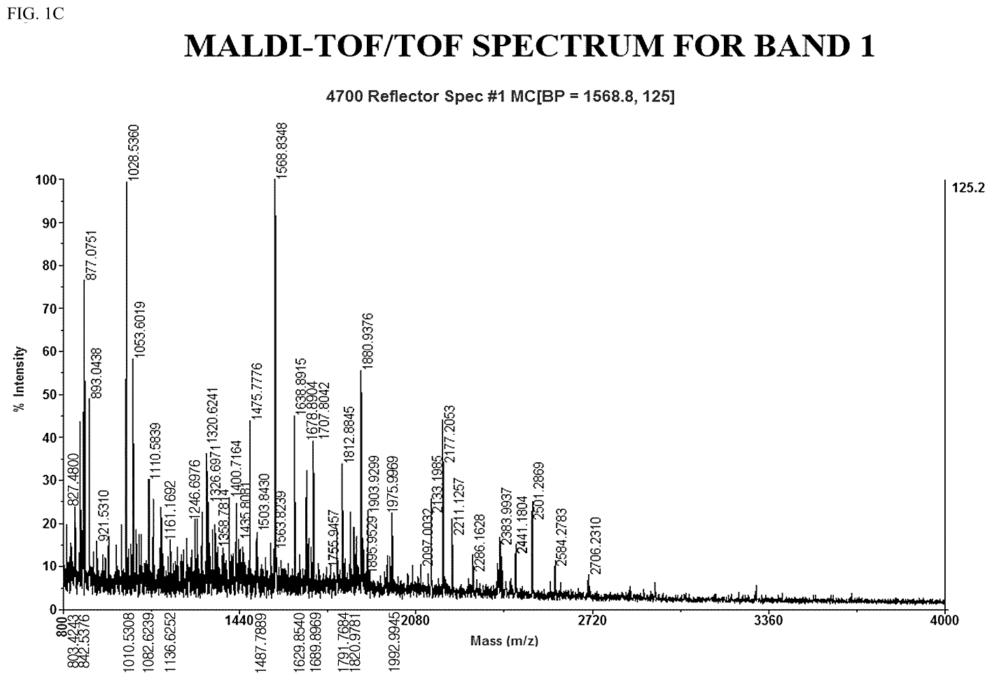

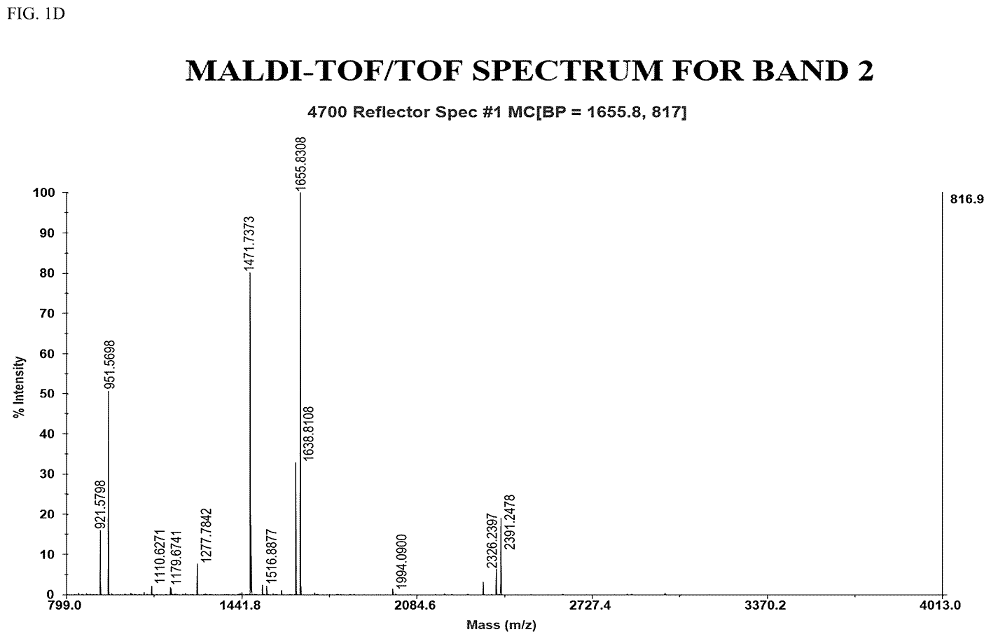

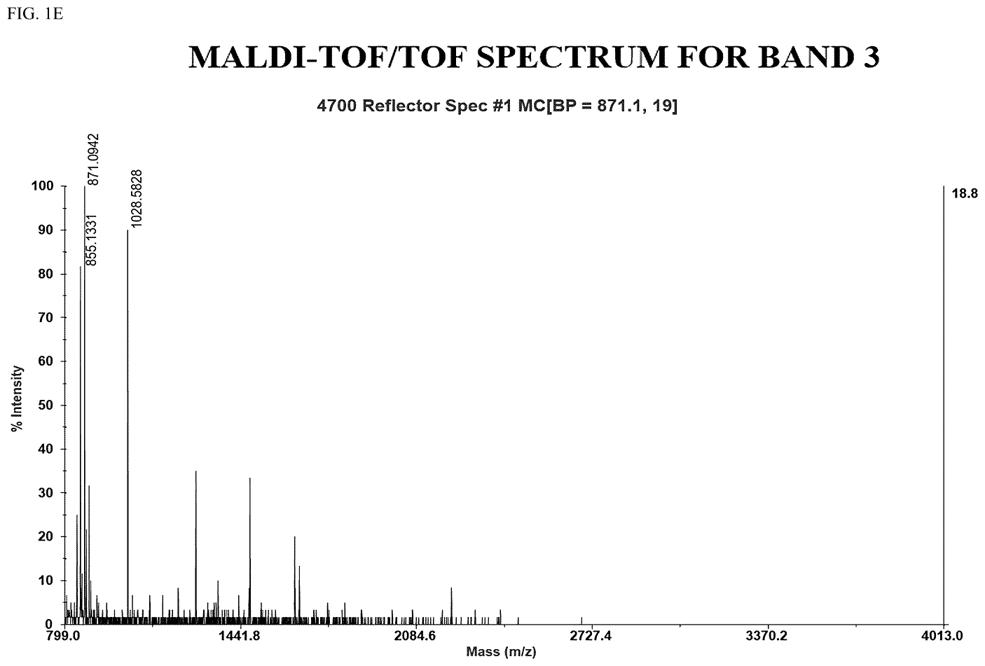

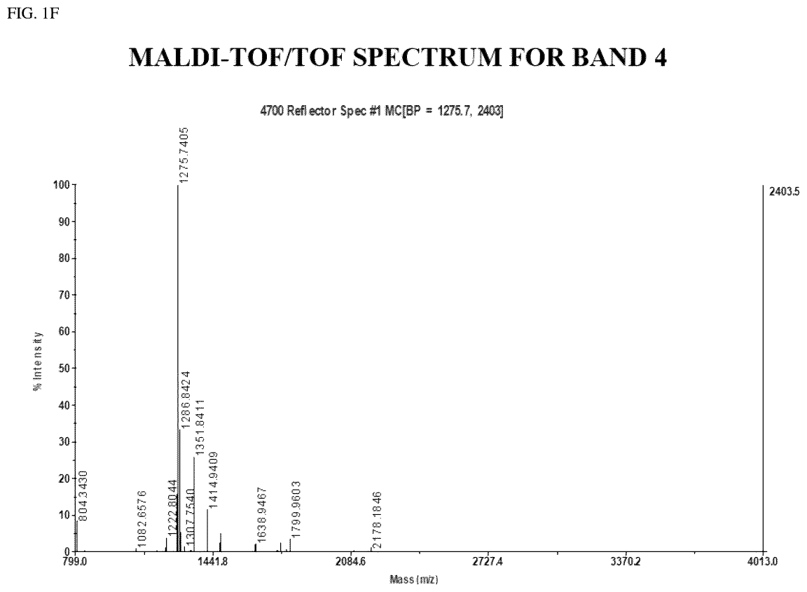

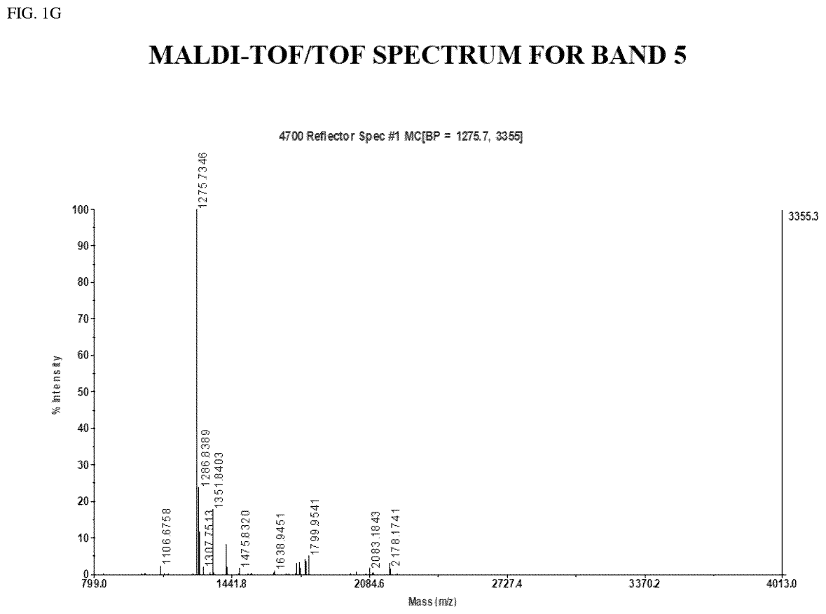

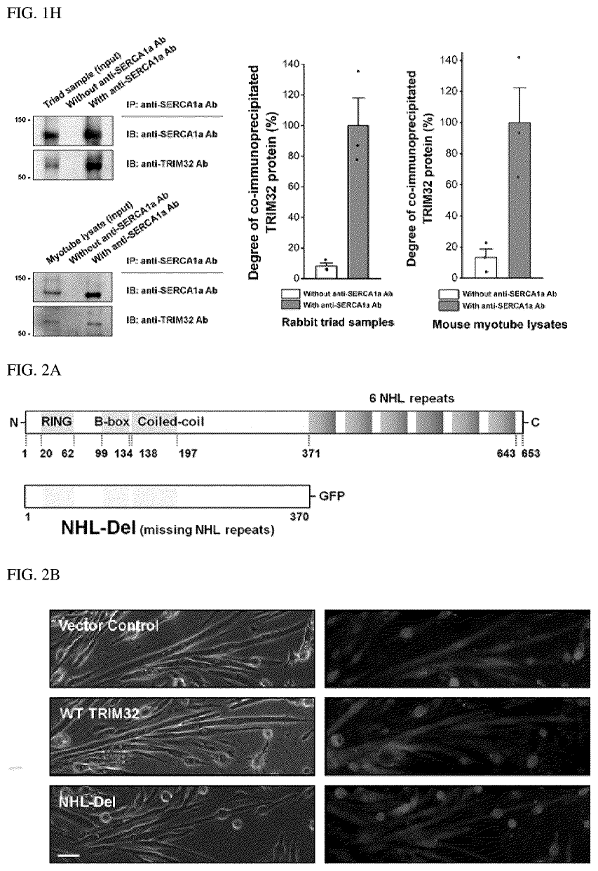

### Example 1: Confirmation of Binding Reaction of TRIM32 and NHL Repeats with SERCA1a (FIG. 1)

To search for a protein that binds to the NHL repeat of TRIM32 in skeletal muscle, the NHL repeat was prepared as GST-NHL (in an N-terminal GST-tagged form) and expressed in E. coli (FIG. 1A). Successful expression of the GST-NHL protein transformed and expressed in E. coli (DH5α) was confirmed through Coomassie Blue staining (left of FIG. 1B). GST-NHL was purified by attaching it to GST beads, and then the degree of purification of GST-NHL was determined through Coomassie Blue staining (center of FIG. 1B). Thereafter, a binding reaction of GST-NHL with the triad sample obtained from the rabbit skeletal muscle was performed, and then the expression of GST-NHL was determined through Coomassie Blue staining (right of FIG. 1B). Here, GST was used as a negative control. Five triad proteins that appear to bind to GST-NHL are marked as Bands 1 to 5. The identity of each of the five proteins was confirmed by MALDI-TOF/TOF mass spectrometry (FIGS. 1C to 1G) and a database (Table 3), and Band 1 was confirmed to be SERCA1a.

TABLE 3

| Band | | GenBank | | |
|---|---|---|---|---|
| # | Protein name | accession code | Species | Matching score |
| 1 | Sarcoplasmic/endoplasmic | NM_001089318 | Rabbit | 103 |
| | reticulum calcium ATPase | | | |
| | 1a (SERCA1a) | | | |
| 2 | TRIM32 | XP_002720557.1 | Rabbit | 180 |
| 3 | No matched signal | | | |
| 4 | TEM-1 Beta-lactamase | ACU00830.1 | E. Coli | 276 |
| 5 | TEM-1b Beta-lactamase | ADB90406.1 | E. Coli | 375 |

Co-immunoprecipitation was performed using a SERCA1a antibody and the triad sample obtained from the rabbit skeletal muscle, and the resulting sample was subjected to immunoblotting analysis using SERCA1a and TRIM32 antibodies (top of FIG. 1H). Co-immunoprecipitation was performed using a lysis solution sample of mouse myotubes and a SERCA1a antibody, and the resulting sample was subjected to immunoblotting analysis using SERCA1a and TRIM32 antibodies (bottom of FIG. 1H). The value obtained by adding the antibody (Without anti-SRECA1a Ab) was set to 1, and a relative change in the value was calculated as a normalized ratio, and shown as a bar graph on the right of FIG. 1H. IB, IP, or Ab refers to an immunoblotting experiment, a co-immunoprecipitation experiment, or an antibody, respectively, and * indicates a significant difference compared to “Without anti-SRECA1a Ab” (p<0.05).

As a result, it was confirmed that TRIM32 binds to SERCA1a through the NHL repeat in both the rabbit skeletal muscle tissue and the mouse skeletal muscle cells, indicating that the binding of SERCA1a and TRIM32 through the NHL repeat is a common phenomenon.

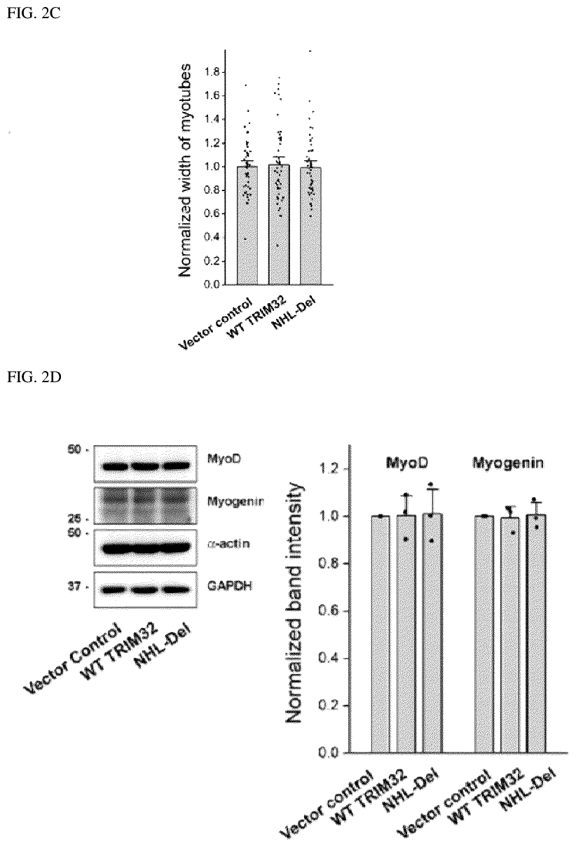

### Example 2: Comparison of Expression and Differentiation States of Wild-Type TRIM32 or NHL-Del in Mouse Myotubes (FIG. 2)

The expression of a vector (vector control), wild-type TRIM32, or NHL-Del in myotubes was confirmed by immunochemistry (FIGS. 2A and 2B). The characteristics of mouse myotubes expressing wild-type TRIM32 or NHL-Del are shown in Table 4. The width of the thickest part of the myotubes was measured and analyzed by comparison with the control. The number of experiments used for analysis and statistics and the resulting values are shown in parentheses in Table 4.

TABLE 4

| | Vector control | WT TRIM32 | NHL-Del |
|---|---|---|---|
| Width of myotubes | 1.00 ± 0.03 | 1.01 ± 0.04 | 0.99 ± 0.04 |
| | (50 myotubes) | (50 myotubes) | (50 myotubes) |
| Releasable Ca²⁺ | 1.00 ± 0.15 | 1.48 ± 0.18 * | 0.99 ± 0.15 |
| level from SR | (72 myotubes | (82 myotubes | (81 myotubes |
| | from 25 wells) | from 18 wells) | from 22 wells) |
| Caffeine response | 1.00 ± 0.07 | 1.22 ± 0.09 | 0.97 ± 0.07 |
| | (85 myotubes | (58 myotubes | (63 myotubes |
| | from 63 wells) | from 50 wells) | from 61 wells) |
| KCl response | 1.00 ± 0.06 | 1.27 ± 0.11 * | 0.94 ± 0.06 |
| | (85 myotubes | (56 myotubes | (61 myotubes |
| | from 52 wells) | from 36 wells) | from 47 wells) |

The expression levels of MyoD and myogenin proteins, which are differentiation marker proteins that show the degree of differentiation of myotubes, were determined through immunoblotting analysis, and the expression level of a-actin, which is a structural protein in all cells, was confirmed (unchanged). A GAPDH protein was used as a loading control whose value was set to 1, and a relative change in the value was calculated as a normalized ratio, and shown as a bar graph (FIGS. 2C and 2D). Three independent experiments were performed per protein, and values are expressed as the mean±SE from the three independent experiments. The expression levels of the proteins were normalized to the average value of the vector control. The number of experiments used in analysis and statistics and the resulting values are shown in Table 5. * indicates a significant difference compared to the control (p<0.05).

TABLE 5

| | Vector control | WT TRIM32 | NHL-Del |
|---|---|---|---|
| MyoD | 1.00 ± 0.00 | 1.00 ± 0.06 | 1.01 ± 0.07 |
| Myogenin | 1.00 ± 0.00 | 0.99 ± 0.03 | 1.01 ± 0.03 |
| RyR1 | 1.00 ± 0.00 | 0.98 ± 0.05 | 1.03 ± 0.05 |
| DHPR | 1.00 ± 0.00 | 1.01 ± 0.04 | 1.02 ± 0.03 |
| SERCA1a | 1.00 ± 0.00 | 0.97 ± 0.03 | 0.98 ± 0.03 |
| TRIM32 | 1.00 ± 0.00 | 1.01 ± 0.06 | 0.99 ± 0.04 |
| CASQ1 | 1.00 ± 0.00 | 1.02 ± 0.02 | 0.99 ± 0.02 |
| MG53 | 1.00 ± 0.00 | 1.02 ± 0.03 | 1.02 ± 0.04 |
| α-actin | 1.00 ± 0.00 | 1.02 ± 0.06 | 1.01 ± 0.02 |

As a result, it was confirmed that the wild-type TRIM32 or NHL-Del was successfully expressed in the mouse myotubes, and that their expression did not affect the expression level of the myotubes.

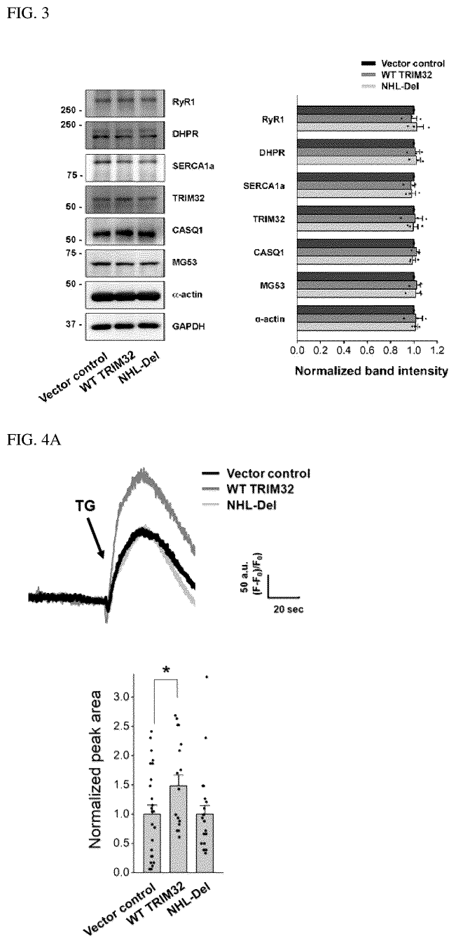

### Example 3: Comparison of Expression Levels of Key Proteins That Mediate Skeletal Muscle Contraction and Relaxation in Mouse Myotubes Expressing Wild-Type TRIM32 or NHL-Del (FIG. 3)

To compare the expression levels of RyR1, DHPR, SERCA1a, TRIM32 (existing in the art), MG53, and CASQ1, which are key proteins that mediate skeletal muscle contraction and relaxation in mouse myotubes expressing wild-type TRIM32 or NHL-Del, immunoblotting analysis was performed. The expression level of a-actin, which is a structural protein in all the cells, was confirmed (unchanged). A GAPDH protein was used as a loading control whose value was set to 1, and a relative change in the value was calculated as a normalized ratio. Three independent experiments were performed per protein, and values are expressed as the mean±SE from the three independent experiments. The expression levels of the proteins were normalized to the average value of the vector control. The number of experiments used for analysis and statistics and the resulting values are shown in Table 5 of Example 2. * indicates a significant difference compared to the control (p<0.05).

As a result, it was confirmed that the expression of wild-type TRIM32 or NHL-Del in the mouse myotubes did not affect the expression levels of the key proteins that mediate the contraction and relaxation of skeletal muscle. This indicates that the phenomenon discovered through this study is caused by the expression of TRIM32 or NHL-Del.

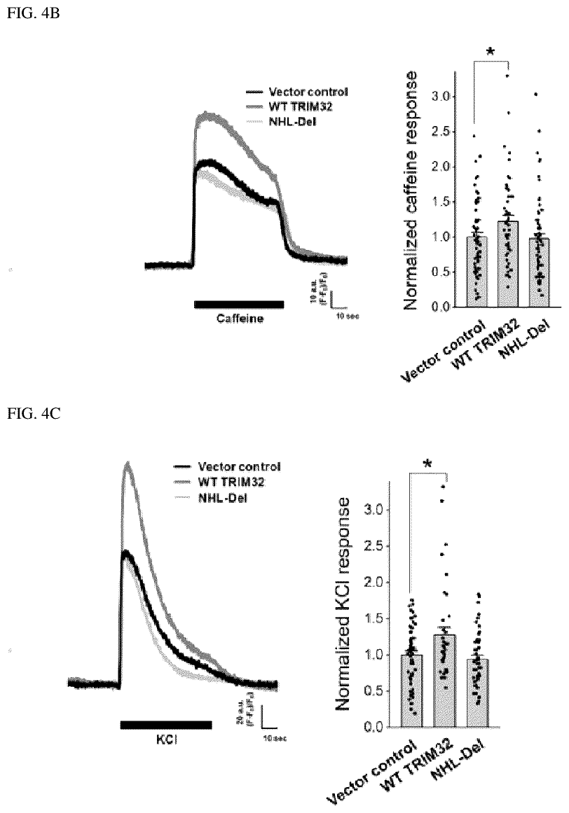

### Example 4: Comparison of Amount of Calcium in Sarcoplasmic Reticulum and Degree of Calcium Transport for Skeletal Muscle Contraction in Mouse Myotubes Expressing Wild-Type TRIM32 or NHL-Del (FIG. 4)

The amount of calcium stored in the sarcoplasmic reticulum was indirectly measured and compared in mouse myotubes expressing wild-type TRIM32 or NHL-Del by thapsigargin (TG) treatment (FIG. 4A). Also, mouse myotubes expressing wild-type TRIM32 or NHL-Del were treated with caffeine, which is a direct/specific activator of ryanodine receptor 1 (RyR1) serving as an internal calcium channel to measure the relative degree of calcium release from the sarcoplasmic reticulum into the cytosol through RyRI for skeletal muscle contraction (FIG. 4B). Thereafter, the relative amount of the released calcium was measured by treating mouse myotubes expressing wild-type TRIM32 or NHL-Del with KCl, which is a cell membrane depolarizing agent, to induce the release of calcium from the sarcoplasmic reticulum into the cytosol for skeletal muscle contraction (FIG. 4C). * indicates a significant difference compared to the control (p<0.05). The number of experiments used for analysis and statistics and the resulting values are shown in parentheses in Table 4 of Example 2.

As a result, it was confirmed that the wild-type TRIM32 increased both the amount of calcium in the sarcoplasmic reticulum and the response to caffeine and KCl (i.e., indicating the degree of calcium transport to cause contraction during skeletal muscle contraction). However, these phenomena are eliminated by deletion of the NHL repeat (i.e., NHL-Del without binding to SERCA1a due to the absence of the SERCA1a binding site). Therefore, it can be seen that TRIM32 mediates an increase in the amount of calcium in the sarcoplasmic reticulum for skeletal muscle contraction and the degree of calcium transport for contraction during skeletal muscle contraction by binding to SERCA1a through its own NHL repeat.

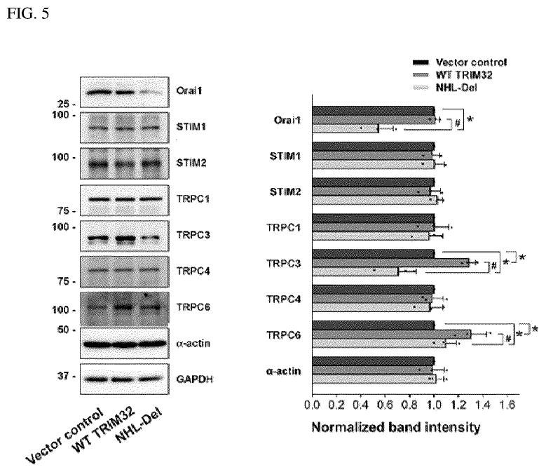

### Example 5: Comparison of Expression Levels of Key Proteins That Mediate External Calcium Influx in Mouse Myotubes Expressing Wild-Type TRIM32 or NHL-Del (FIG. 5)

To compare the expression levels of Orai1, STIM1, STIM2, TRPC1, TRPC3, TRPC4, and TRPC6, which are key proteins that mediate external calcium influx as a calcium transport pathway required for skeletal muscle contraction in mouse myotubes expressing wild-type TRIM32 or NHL-Del, immunoblotting analysis was performed. The expression level of a-actin, which is a structural protein in all cells, was confirmed (unchanged). A GAPDH protein was used as a loading control. The value of the control was set to 1, and a relative change in the value was expressed as a normalized ratio. The number of experiments used for analysis and statistics and the resulting values are shown in Table 6. * indicates a significant difference compared to the control (p<0.05).

TABLE 6

| | Vector control | WT TRIM32 | NHL-Del |
|---|---|---|---|
| Orai1 | 1.00 ± 0.00 | 1.01 ± 0.02 | 0.54 ± 0.08 *^{, #} |
| STIM1 | 1.00 ± 0.00 | 0.99 ± 0.04 | 1.01 ± 0.05 |
| STIM2 | 1.00 ± 0.00 | 0.97 ± 0.06 | 1.03 ± 0.03 |
| TRPC1 | 1.00 ± 0.00 | 1.00 ± 0.08 | 0.96 ± 0.07 |
| TRPC3 | 1.00 ± 0.00 | 1.29 ± 0.04 * | 0.71 ± 0.10 *^{, #} |
| TRPC4 | 1.00 ± 0.00 | 0.98 ± 0.06 | 0.97 ± 0.07 |
| TRPC6 | 1.00 ± 0.00 | 1.30 ± 0.09 * | 1.10 ± 0.06 *^{, #} |
| α-actin | 1.00 ± 0.00 | 0.99 ± 0.06 | 1.02 ± 0.04 |

As a result, it was confirmed that the expression of NHL-Del in the mouse myotubes (i.e., a cell model manifesting an LGMD2H disease) was accompanied by a decrease in the expression of Orai1 and TRPC3, which mediate external calcium influx as a calcium transport pathway required for skeletal muscle contraction, and an increase in the expression of TRPC6. Because this phenomenon is completely different from that caused by wild-type TRIM32, it was confirmed that the proteins were able to be used as diagnostic markers to diagnose LGMD2H in patients.

According to the present invention, information for the diagnosis of limb-girdle muscular dystrophy type 2H can be provided, and drugs for the prevention or treatment of limb-girdle muscular dystrophy type 2H can be screened.

Although the present invention has been described with reference to the above-described examples and experimental examples, the examples and experimental examples are merely provided for illustrative purposes, and those skilled in the art will appreciate that various modifications and other equivalent embodiments may be made therein without departing from the scope of the present invention. Thus, the true technical scope of protection of the present invention should be defined by the appended claims.

What is claimed is:

1. A method of providing information for the diagnosis of limb-girdle muscular dystrophy type 2H, comprising:
measuring the expression level of the mRNA or protein of one or more genes selected from the group consisting of Orai1, TRPC3, and TRPC6 in a biological sample isolated from a subject.

2. The method of claim 1, further comprising:
judging that the subject has developed limb-girdle muscular dystrophy type 2H or is at high risk of developing limb-girdle muscular dystrophy type 2H when the expression level of Orai1 and/or TRPC3 decreases compared to the expression level in the normal control sample; or when the expression level of TRPC6 increases compared to the expression level in the normal control sample.

3. A method of screening a drug for the treatment of limb-girdle muscular dystrophy type 2H, comprising:
treating a biological sample, which is isolated from a subject suspected of having limb-girdle muscular dystrophy type 2H, with a candidate material, and judging whether the candidate material increases or decreases the expression of the mRNA or protein of one or more genes selected from the group consisting of Orai1, TRPC3, and TRPC6 in skeletal muscle cells.

4. The method of claim 3, wherein the candidate material is judged to be a therapeutic agent for limb-girdle muscular dystrophy type 2H when the candidate material increases the expression of the mRNA or protein of the Orai1 and/or TRPC3 genes in skeletal muscle cells.

5. The method of claim 3, wherein the candidate material is judged to be a therapeutic agent for limb-girdle muscular dystrophy type 2H when the candidate material decreases the expression of the mRNA or protein of the TRPC6 gene in skeletal muscle cells.


SEQUENCE LISTING (4 sequences)

SEQ ID NO: 1 - DNA, 22 bp, unidentified
  source 1..22: mol_type = genomic DNA
cggaattcatgttcaatcttcc

SEQ ID NO: 2 - DNA, 24 bp, unidentified
  source 1..24: mol_type = genomic DNA
cctcgagtcatggggtggaatatc

SEQ ID NO: 3 - DNA, 24 bp, unidentified
  source 1..24: mol_type = genomic DNA
gactcagatctcgaggccaccatg

SEQ ID NO: 4 - DNA, 21 bp, unidentified
  source 1..21: mol_type = genomic DNA
cgggatcccattcctggagtg